(19) United States
(12) Patent Application Publication
(10) Pub. No.: US 2022/0291240 A1
(43) Pub. Date: Sep. 15, 2022

(54) BIOMARKERS FOR NEURODEGENERATIVE DISORDERS

(71) Applicant: The Johns Hopkins University, Baltimore, MD (US)

(72) Inventors: Ted M. Dawson, Baltimore, MD (US); Liana Rosenthal, Baltimore, MD (US); Saurav Brahmachari, Baltimore, MD (US); Tae-In Kam, Baltimore, MD (US); Valina L. Dawson, Baltimore, MD (US)

(21) Appl. No.: 17/633,917

(22) PCT Filed: Aug. 26, 2020
(86) PCT No.: PCT/US2020/048045

§ 371 Date: Feb. 8, 2022

Related U.S. Application Data
(60) Provisional application No. 62/892,180, filed on Aug. 27, 2019.

(51) Int. Cl. G01N 33/68

(52) U.S. Cl. CPC G01N 33/6896; G01N 2800/2835; G01N 2440/14

(57) ABSTRACT

The invention relates to methods for diagnosis, monitoring progression, and treatment of neurodegenerative disorders. In particular, biomarkers for diagnosis, monitoring progression, and treatment of neurodegenerative disorders are provided. In some embodiments, methods for diagnosis, monitoring progression, and treatment of synucleinopathies and related disorders are provided.

### CROSS-REFERENCE TO RELATED APPLICATIONS

This application claims benefit of priority under 35 U.S.C. § 119(e) of U.S. Ser. No. 62/892,180 filed Aug. 27, 2019, the entire contents of which is incorporated herein by reference in its entirety.

### STATEMENT OF GOVERNMENT SUPPORT

This invention was made with government support under Grant No. NS038377, NS082133, and NS097049 awarded by the National Institutes of Health. The government has certain rights in the invention.

### TECHNICAL FIELD

The present invention generally relates to the use of biomarkers for the diagnosis, monitoring, and treatment of neurodegenerative diseases.

### BACKGROUND

Parkinson's disease (PD) continues to be diagnosed based solely on clinical history and examination as there is no blood test or imaging that can diagnose the disease. Many patients therefore go upwards of 2 years from symptom onset to diagnosis and movement disorder physicians are incorrect about the diagnosis as much as 10% of the time, with the most common error being the inability to differentiate between PD and other forms of Parkinsonism. Further, treatment options remain suboptimal with unacceptable side effects including dyskinesias, motor fluctuations, and ultimately even hallucinations and cognitive impairment due to the interactions of the medication with the diseased brain. Most concerning, no current treatment options are disease modifying. The subsequent motor and cognitive progression significantly increases PD patients' morbidity and mortality compared to the general population.

A good PD biomarker has the potential to fundamentally change how the disease is diagnosed and managed. Diagnostic and progression biomarkers would improve the speed and accuracy of diagnosis, improve prognostication by treating physicians and enhance the ability to offer disease modifying therapies earlier in the disease course. In addition, a diagnostic marker could be used to enhance cohort selection for clinical trials and a progression marker could serve as surrogate endpoints for any investigation, therefore improving trial efficiency.

### SUMMARY OF THE INVENTION

The present invention is based on the discovery that biomarkers can be used for diagnosis, monitoring progression, and treatment of neurodegenerative diseases.

The invention provides methods for diagnosis, monitoring progression, and treatment of neurodegenerative diseases. In particular, biomarkers that can be used in methods for diagnosis, monitoring progression, and treatment of neurodegenerative diseases are provided. In some embodiments, methods for diagnosis, monitoring progression, and treatment of synucleinopathies and related diseases are provided.

Described herein, in some embodiments, are methods of diagnosing a neurodegenerative disease in a subject comprising: (a) determining an expression level, a phosphorylation level, and/or an activation level of one or more biomarkers in a biological sample obtained from a subject suspected of having a neurodegenerative disease; (b) determining that the subject suffers from a neurodegenerative disease when the expression level, the phosphorylation level, and/or the activation level of the one or more biomarkers in the biological sample is altered relative to the expression level, the phosphorylation level, and/or the activation level of the one or more biomarkers in a biological sample of a control subject; and (c) providing a report of the determination that the subject suffers from a neurodegenerative disease for selection of a treatment of the subject. In some aspects, the one or more biomarkers is a nucleic acid or a protein. In some aspects, the one or more biomarkers is a molecule of the c-Abl pathway. In some aspects, the molecule of the c-Abl pathway is a protein. In some aspects, the molecule is c-Abl, α-synuclein, parkin, AIMP2, PARIS (ZNF746), PARP1, PAR, or a combination thereof. In some aspects, the altered expression level, the altered phosphorylation level, and/or the altered activation level of the one or more biomarkers is selected from the group consisting of: (a) the level of c-Abl phosphorylation is increased; (b) the level of c-Abl activation is increased; (c) the level of α-synuclein phosphorylation is increased; (d) the level of parkin phosphorylation is increased; (e) the level of parkin inactivation is increased; (f) the expression level of AIMP2 is increased; (g) the level of AIMP2 phosphorylation is increased; (h) the expression level of PARIS (ZNF746) is increased; (i) the level of PARP1 activation is increased; and (j) the level of PAR is increased; or a combination thereof. In some aspects, phosphorylation of c-Abl is selected from tyrosine 245 (Y245); tyrosine 412 (Y412); and tyrosine 245 (Y245) and tyrosine 412 (Y412). In some aspects, phosphorylation of α-synuclein is selected from tyrosine 39 (Y39); serine 129 (S129); and tyrosine 39 (Y39) and serine 129 (S129). In some aspects, phosphorylation of parkin is on tyrosine 143 (Y143). In some aspects, phosphorylation of AIMP2 is on tyrosine 25 (Y25). In some aspects, phosphorylation comprises any combination of: (a) phosphorylation of c-Abl is selected from tyrosine 245 (Y245); tyrosine 412 (Y412); and tyrosine 245 (Y245) and tyrosine 412 (Y412); (b) phosphorylation of α-synuclein is selected from tyrosine 39 (Y39); serine 129 (S129); and tyrosine 39 (Y39) and serine 129 (S129); (c) phosphorylation of parkin is on tyrosine 143 (Y143); and (d) phosphorylation of AIMP2 is on tyrosine 25 (Y25). In some aspects, the biological sample is a blood sample, a plasma sample, or a serum sample. In some aspects, the biological sample comprises exosomes. In some aspects, the exosomes include a neuronal marker. In some aspects, the neuronal marker is L1CAM. In some aspects, the neurodegenerative disease is a synucleinopathy. In some aspects, the synucleinopathy is Parkinson's disease, dementia with Lewy bodies, multiple system atrophy (MSA), or a neuraxonal dystrophy. In some aspects, the control subject is healthy, does not suffer from a neurodegenerative disease, or suffers from Parkinsonism, Parkinson's-like disease, or a tauopathy. In some aspects, methods of diagnosing a neurodegenerative disease described herein further comprise determining that the neurodegenerative disease is a synucleinopathy as distinguished from a tauopathy.

Described herein, in some embodiments, are methods for monitoring progression of a neurodegenerative disease in a subject comprising: (a) determining an expression level, a phosphorylation level, and/or an activation level of one or more biomarkers in a biological sample obtained from a subject suspected of having a neurodegenerative disease; wherein the expression level, the phosphorylation level, and/or the activation level of the one or more biomarkers is altered upon progression of the neurodegenerative disease and when sampled at a later time point relative to an earlier time point of the neurodegenerative disease; and (b) providing a report of the altered expression level, the altered phosphorylation level, and/or the altered activation level of the one or more biomarkers for selection of a treatment of the subj ect. In some aspects, the one or more biomarkers is a nucleic acid or a protein. In some aspects, the one or more biomarkers is a molecule of the c-Abl pathway. In some aspects, the molecule of the c-Abl pathway is a protein. In some aspects, the molecule is c-Abl, α-synuclein, parkin, AIMP2, PARIS (ZNF746), PARP1, PAR, or a combination thereof. In some aspects, the altered expression level, the altered phosphorylation level, and/or the altered activation level of the one or more biomarkers is selected from the group consisting of: (a) the level of c-Abl phosphorylation is increased; (b) the level of c-Abl activation is increased; (c) the level of α-synuclein phosphorylation is increased; (d) the level of parkin phosphorylation is increased; (e) the level of parkin inactivation is increased; (f) the expression level of AIMP2 is increased; (g) the level of AIMP2 phosphorylation is increased; (h) the expression level of PARIS (ZNF746) is increased; (i) the level of PARP1 activation is increased; and (j) the level of PAR is increased; or a combination thereof. In some aspects, phosphorylation of c-Abl is selected from tyrosine 245 (Y245); tyrosine 412 (Y412); and tyrosine 245 (Y245) and tyrosine 412 (Y412). In some aspects, phosphorylation of α-synuclein is selected from tyrosine 39 (Y39); serine 129 (S129); and tyrosine 39 (Y39) and serine 129 (S129). In some aspects, phosphorylation of parkin is on tyrosine 143 (Y143). In some aspects, phosphorylation of AIMP2 is on tyrosine 25 (Y25). In some aspects, phosphorylation comprises any combination of: (a) phosphorylation of c-Abl is selected from tyrosine 245 (Y245); tyrosine 412 (Y412); and tyrosine 245 (Y245) and tyrosine 412 (Y412); (b) phosphorylation of α-synuclein is selected from tyrosine 39 (Y39); serine 129 (S129); and tyrosine 39 (Y39) and serine 129 (S129); (c) phosphorylation of parkin is on tyrosine 143 (Y143); and (d) phosphorylation of AIMP2 is on tyrosine 25 (Y25). In some aspects, the biological sample is a blood sample, a plasma sample, or a serum sample. In some aspects, the biological sample includes exosomes. In some aspects, the exosomes include a neuronal marker. In some aspects, the neuronal marker is L1CAM. In some aspects, the neurodegenerative disease is a synucleinopathy. In some embodiments, the synucleinopathy is Parkinson's disease, dementia with Lewy bodies, multiple system atrophy (MSA), or a neuraxonal dystrophy. In some embodiments, the expression level, the phosphorylation level, and/or the activation level of the one or more biomarkers is determined at two or more time points.

Described herein, in some embodiments, are methods of treating a neurodegenerative disease in a subject comprising administering an inhibitor of the c-Abl pathway to the subject when the expression level, the phosphorylation level, and/or the activation level of one or more biomarkers of the c-Abl pathway in a biological sample obtained from the subject is altered relative to the expression level, the phosphorylation level, and/or the activation level of the one or more biomarkers in a biological sample of a control subject. In some aspects, the one or more biomarkers is a nucleic acid or a protein. In some aspects, the one or more biomarkers is c-Abl, α-synuclein, parkin, AIMP2, PARIS (ZNF746), PARP1, PAR, or a combination thereof. In some aspects, the altered expression level, the altered phosphorylation level, and/or the altered activation level of the one or more biomarkers is selected from the group consisting of: (a) the level of c-Abl phosphorylation is increased; (b) the level of c-Abl activation is increased; (c) the level of α-synuclein phosphorylation is increased; (d) the level of parkin phosphorylation is increased; (e) the level of parkin inactivation is increased; (f) the expression level of AIMP2 is increased; (g) the level of AIMP2 phosphorylation is increased; (h) the expression level of PARIS (ZNF746) is increased; (i) the level of PARP1 activation is increased; and (j) the level of PAR is increased; or a combination thereof. In some aspects, phosphorylation of c-Abl is selected from tyrosine 245 (Y245); tyrosine 412 (Y412); and tyrosine 245 (Y245) and tyrosine 412 (Y412). In some aspects, phosphorylation of α-synuclein is selected from tyrosine 39 (Y39); serine 129 (S129); and tyrosine 39 (Y39) and serine 129 (S129). In some aspects, phosphorylation of parkin is on tyrosine 143 (Y143). In some aspects, phosphorylation of AIMP2 is on tyrosine 25 (Y25). In some aspects, phosphorylation comprises any combination of: (a) phosphorylation of c-Abl is selected from tyrosine 245 (Y245); tyrosine 412 (Y412); and tyrosine 245 (Y245) and tyrosine 412 (Y412); (b) phosphorylation of α-synuclein is selected from tyrosine 39 (Y39); serine 129 (S129); and tyrosine 39 (Y39) and serine 129 (S129); (c) phosphorylation of parkin is on tyrosine 143 (Y143); and (d) phosphorylation of AIMP2 is on tyrosine 25 (Y25). In some aspects, the biological sample is a blood sample, a plasma sample, or a serum sample. In some aspects, the biological sample includes exosomes. In some aspects, the exosomes comprises a neuronal marker. In some aspects, the neuronal marker is L1CAM. In some aspects, the neurodegenerative disease is a synucleinopathy. In some aspects, the synucleinopathy is Parkinson's disease, dementia with Lewy bodies, multiple system atrophy (MSA), or a neuraxonal dystrophy. In some aspects, the control subject is healthy, does not suffer from a neurodegenerative disease, or suffers from Parkinsonism, Parkinson's-like disease, or a tauopathy. In some aspects, the inhibitor of the c-Abl pathway is a kinase inhibitor. In some aspects, the kinase inhibitor is a c-Abl inhibitor. In some aspects, the inhibitor of the c-Abl pathway is a PARP inhibitor. In some aspects, the PARP inhibitor is a PARP1 inhibitor. In some aspects, methods of treatment of neurodegenerative diseases described herein further comprise determining that the neurodegenerative disease is a synucleinopathy as distinguished from a tauopathy.

### BRIEF DESCRIPTION OF THE DRAWINGS

FIG. 1 illustrates the c-Abl pathway.

FIGS. 2A-2E illustrate elevation of molecules of the c-Abl pathway in serum-derived L1CAM+ exosomes of Parkinson's disease (PD) patients. (FIG. 2A) Representative immunoblots of pY245 c-Abl, c-Abl, PARIS, pY25 AIMP2, AIMP2, pY39 α-syn, pS129 α-syn, α-syn, hemoglobin β, L1CAM, CD9, and CD81 in serum-derived L1CAM+ exosomes from PD patients and control subjects. (FIG. 2B) Quantifications of pY245 c-Abl normalized to c-Abl, and quantifications pY245 c-Abl and c-Abl normalized to L1CAM. (FIG. 2C) Quantifications of PARIS normalized to L1CAM. (FIG. 2D) Quantifications of pY25 AIMP2 and AIMP2 normalized to L1CAM. (FIG. 2E) Quantifications of pY39 α-syn normalized to α-syn, and pY39 α-syn and α-syn normalized to L1CAM. (n: Control=8, PD=10). Statistical significance was determined by unpaired two-tailed t-test. Quantified data are expressed as mean±s.e.m. *p<0.05, **p<0.01, ***p<0.001. ns, not significant.

FIGS. 3A-3B illustrate elevated levels of c-Abl activation and pY39 α-synuclein in exosomes of MSA patients, but not in exosomes of PSP and CBS patients. No elevated levels of PARIS and AIMP2 were observed. (FIG. 3A) Representative immunoblots of pY245 c-Abl, c-Abl, pY39 α-syn, α-syn, PARIS, AIMP2, L1CAM, CD9, and CD81 in serum-derived L1CAM+ exosomes from MSA, PSP, and CBS patients and control subjects. (FIG. 3B) Quantifications of pY245 c-Abl normalized to c-Abl, pY245 c-Abl and c-Abl normalized to L1CAM, pY39 α-syn normalized α-syn, and pY39 α-syn and α-syn normalized to L1CAM (n: Control=3, MSA=2, PSP=3, CBS=3). Statistical significance was determined by 1-way ANOVA with Sidak's post-test of multiple comparison. Quantified data are expressed as mean ±s.e.m. *p<0.05, **p<0.01, ***p<0.001.

FIGS. 4A-4E illustrate elevated molecules of c-Abl pathway in serum-derived L1CAM+ exosomes of PD patients. (FIG. 4A) Representative immunoblots of pY245 c-Abl, c-Abl, pY137 PARIS, PARIS, pY25 AIMP2, AIMP2, pY39 α-syn, α-syn, L1CAM, CD81, and CD9 in serum-derived L1CAM+ exosomes from PD patients and control subjects. (FIG. 4B) Quantifications of pY245 c-Abl normalized to c-Abl, and L1CAM. (FIG. 4C) Quantifications of pY137 PARIS, and PARIS normalized to L1CAM. (FIG. 4D) Quantifications of pY25 AIMP2 and AIMP2 normalized to L1CAM. (FIG. 4E) Quantifications of pY39 α-syn normalized to α-syn, and to L1CAM. *p<0.05, **p<0.01, ***p<0.001. ns, not significant.

FIGS. 5A-5B illustrate an MSD-based quantitative immunoassays showing elevation of pY245 c-Abl and pY39 α-synuclein in the serum-derived L1CAM+ exosomes of PD patients. (FIG. 5A) bar diagrams representing concentrations of pY245 c-Abl measured by electrochemiluminescence. (FIG. 5B) bar diagrams representing concentrations of pY39 α-syn measured by electrochemiluminescence. *p<0.05, ***p<0.001.

FIG. 6 illustrates an MSD-based quantitative immunoassay revealing increased PAR levels in the serum-derived L1CAM+ exosomes of PD patients, as measured by ECL immunoassay. *p<0.05.

FIG. 7 illustrates an MSD-based quantitative immunoassay for α-synuclein in the serum-derived L1CAM+ exosomes of PD cases and age-matched healthy controls as measured by ECL immunoassay. ns, not significant.

FIGS. 8A-8C illustrate the characterization of serum-derived neuronally enriched exosomes. (FIG. 8A) Concentrations and sizes of serum-derived L1CAM-enriched EVs as analyzed by Spectradyne's nCS1. (FIG. 8B) immunoblot of canonical exosomal markers and the neuronal markers in the L1CAM-enriched neuronal EVs. (FIG. 8C) immunoblot of canonical exosomal markers and the neuronal markers in the CD81+ total EVs.

FIGS. 9A-D illustrate the characterization of serum derived total exosomes isolated by a high throughput size-exclusion-based EV isolation method. (FIG. 9A) Concentrations and sizes of serum-derived EV fraction 1 as analyzed by Spectradyne's nCS1. (FIG. 9B) Concentrations and sizes of serum-derived EV fraction 2 as analyzed by Spectradyne's nCS1. (FIG. 9C) Immunoblots of canonical exosomal markers Alix, TSG101, CD9, and CD81 in the fraction 1 and 2 EV lysates. (FIG. 9D) Immunoblots of c-Abl, α-synuclein, and the neuronal marker L1CAM in the fraction 1 and 2 EV lysates.

### DETAILED DESCRIPTION OF THE INVENTION

The present invention relates to the discovery that biomarkers can be used for diagnosis, monitoring progression, and treatment of neurodegenerative diseases. In particular, methods of the invention utilize biomarkers contained in biological samples for diagnosis, monitoring progression, and treatment of neurodegenerative diseases. Because biological sample such as a biological fluid can be easily collected, methods of the invention are useful in allowing for diagnosis, monitoring progression, and treatment of neurodegenerative diseases without the need for invasive biopsies. Methods of the invention further permit neurodegenerative diseases to be distinguished without the need for invasive procedures, such as distinguishing synucleinopathies from tauopathies, for example.

As used herein, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

“About” as used herein when referring to a measurable value such as an amount, a temporal duration, and the like, is meant to encompass variations of ±20%, or ±10%, or ±5%, or even ±1% from the specified value, as such variations are appropriate for the disclosed methods or to perform the disclosed methods.

Unless defined otherwise, all technical and scientific terms used herein have the same meaning as is commonly understood by one of skill in the art to which this invention belongs.

As used herein, the term “protein” refers to any polymeric chain of amino acids. The terms “peptide” and “polypeptide” are used interchangeably with the term “protein” and also refer to a polymeric chain of amino acids. The term “protein” encompasses native or artificial proteins, protein fragments and polypeptide analogs of a protein sequence. A protein may be monomeric or polymeric. The term “protein” encompasses fragments and variants (including fragments of variants) thereof, unless otherwise contradicted by context.

As used herein, the term “nucleic acid” refers to any deoxyribonucleic acid (DNA) molecule, ribonucleic acid (RNA) molecule, or nucleic acid analogues. A DNA or RNA molecule can be double-stranded or single-stranded and can be of any size. Exemplary nucleic acids include, but are not limited to, chromosomal DNA, plasmid DNA, cDNA, cell-free DNA(cfDNA), mRNA, tRNA, rRNA, siRNA, micro RNA (miRNA or miR), hnRNA. Exemplary nucleic analogues include peptide nucleic acid, morpholino- and locked nucleic acid, glycol nucleic acid, and threose nucleic acid.

As used herein, the term “subject” refers to any individual or patient on which the methods disclosed herein are performed. The term “subject” can be used interchangeably with the term “individual” or “patient.” The subject can be a human, although as will be appreciated by those in the art that the subject may be an animal. Thus, other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject.

As used herein, the terms “sample” and “biological sample” refer to any sample suitable for the methods provided by the present invention. A biological sample can include nucleic acid and protein directly available in the sample, or contained within cells, exosomes, vesicles, and the like present in the biological sample. A biological sample including cells or exosomes used in the present method can be obtained from tissue samples or bodily fluid from a subject, or tissue obtained by a biopsy procedure (e.g., a needle biopsy) or a surgical procedure. In certain aspects, the biological sample of the present invention is a sample of bodily fluid, e.g., cerebral spinal fluid (CSF), blood, serum, plasma, urine, saliva, tears, and ascites, for example. A sample of bodily fluid can be collected by any suitable method known to a person of skill in the art.

As used herein, the term “expression level” refers to the level of a macromolecule in a biological sample, cell, exosome, or extract derived from a biological sample, cell or exosome. A macromolecule can be any polymer. Thus, the term “expression level” can refer to the level of, e.g., proteins or nucleic acids present in the biological sample, synthesized in the cell or present in the cell, exosome, or extract thereof. The term “expression level” can also refer to the level of other macromolecules present in a biological sample, cell, an exosome, or an extract derived from a biological sample, cell or exosome. Macromolecules other than nucleic acids or proteins can include lipids and carbohydrates, for example. Further macromolecules or polymers include, for example, poly (ADP) ribose (PAR) polymer. The terms “expression level” and “level” can be used interchangeably, unless indicated otherwise or contrary to context.

Described herein, in some embodiments, are methods of diagnosing a neurodegenerative disease in a subject comprising (a) determining an expression level, a phosphorylation level, and/or an activation level of one or more biomarkers in a biological sample obtained from a subject suspected of having a neurodegenerative disease; (b) determining that the subject suffers from a neurodegenerative disease when the expression level, the phosphorylation level, and/or the activation level of the one or more biomarkers in the biological sample is altered relative to the expression level, the phosphorylation level, and/or the activation level of the one or more biomarkers in a biological sample of a control subject; and (c) providing a report of the determination that the subject suffers from a neurodegenerative disease for selection of a treatment of the subject.

Described herein, in some embodiments, are methods of monitoring progression of a neurodegenerative disease in a subject comprising: (a) determining an expression level, a phosphorylation level, and/or an activation level of one or more biomarkers in a biological sample obtained from a subject suspected of having a neurodegenerative disease; wherein the expression level, the phosphorylation level, and/or the activation level of the one or more biomarkers is altered upon progression of the neurodegenerative disease and when sampled at a later time point relative to an earlier time point of the neurodegenerative disease; and (b) providing a report of the altered expression level, the altered phosphorylation level, and/or the altered activation level of the one or more biomarkers for selection of a treatment of the subj ect.

Described herein, in some embodiments, are methods of treating a neurodegenerative disease in a subject comprising administering an inhibitor of the c-Abl pathway to the subject when the expression level, the phosphorylation level, and/or the activation level of one or more biomarkers of the c-Abl pathway in a biological sample obtained from the subject is altered relative to the expression level, the phosphorylation level, and/or the activation level of the one or more biomarkers in a biological sample of a control subject.

In some aspects, the one or more biomarkers of the c-Abl pathway in the biological sample obtained from the subject are present and/or expressed in exosomes that can be isolated from the biological sample.

### Exosomes

Exosomes are membrane-bound vesicles. Exosomes can be produced from the endosomal compartment of eukaryotic cells. Exosomes may be found in tissues and in biological fluids, such as blood, serum, plasma, urine, cerebrospinal fluid, ascites, and others. Exosomes can range in size from about 30 nanometers (nm) to several hundred nanometers (nm). Exosomes can contain cell surface proteins, glycoproteins, and lipids, for example. Exosomes can also contain molecules found in the cytosol of cells, such as intracellular proteins, lipids, and nucleic acid. Accordingly, exosomes can contain markers of the cells they are derived from. The exosomes of the methods described herein can contain any cellular marker.

In some embodiments described herein, exosomes are derived from neuronal cells. Exosomes from neuronal cells can have neuronal markers. Exemplary neuronal markers include L1 cell adhesion molecule (L1CAM, L1 protein), synaptophysin, NCAM, gamma-enolase or enolase 2 (NSE), Neuronal Nuclei (NeuN), microtubule-associated protein-2 (MAP-2), tubulin beta III (TUBB3 or TuJ1), doublecortin (DCX), c-fos activation, choline acetyltransferase (ChAT), tyrosine hydroxylase (TH), polysialic acid-neural cell adhesion molecule (PSA-NCAM), neurogenic differentiation 1 (NeuroD or Beta2), tau, calbindin-D28k, calretinin, neurofilament protein (NFP), synaptoporin (SYNPR, SPO), and others. In some embodiments, the exosomes of the methods described herein comprise a neuronal marker. The exosomes of the methods described herein can comprise any neuronal marker. In some embodiments, the neuronal marker is L1CAM.

### L1CAM Neuronal Marker

L1CAM (L1 protein) is located on the neuronal surface throughout the nervous system. L1CAM is conserved in human, mouse, chick, and Drosophila nervous systems, for example. L1CAM is a cell surface glycoprotein that has 1253 amino acids in humans. L1CAM is cell adhesion molecule, promotes cell motility, functions in synaptic plasticity, and regulates signal transduction, among other functions. L1CAM plays a role in neuron-neuron adhesion, neurite fasciculation, outgrowth of neurites, cerebellar granule cell migration, and neurite outgrowth on Schwann cells, for example. The presence of L1CAM in the lipid bilayer of exosomes can indicate that the exosomes have neuronal cell origin. In some embodiments described herein, L1CAM is found in the lipid bilayer of exosomes purified from a biological sample. In some embodiments, the biological sample from which exosomes of neuronal cell origin are purified is a biological fluid such as a blood sample, a serum sample, or a plasma sample.

### Determining Expression Levels, Phosphorylation Levels, and/or Activation Levels of Biomarkers

As described herein, the present invention provides methods for diagnosing a neurodegenerative disease, monitoring progression of a neurodegenerative disease, and treating a neurodegenerative disease. Accordingly, methods of the present invention can include determining expression levels, phosphorylation levels, and/or activation levels of one or more biomarkers. Any cellular protein, nucleic acid, or other macromolecule such as a lipid, carbohydrate, or other polymer can be a biomarker.

Biomarkers of the present invention are present in the biological sample. Biomarkers of the present invention can be in the cytosol of a cell or on the cell surface. Biomarkers of the present invention can also be found in exosomes isolated from a biological sample, including a tissue or biological fluid, as described above. Biomarkers can include a nucleic acid or protein. Any nucleic acid or protein found in the biological sample, in a cell or on a cell's surface, or in an exosomederived therefrom can be a biomarker. Exemplary nucleic acids that can be biomarkers include cfDNA, miRNA, siRNA, and others. Exemplary proteins that can be biomarkers include any protein found in the biological sample, in the cytosol or on the cell surface, or in an exsosome derived therefrom, including glycoproteins and other transmembrane proteins. Other macromolecules or polymers contained in biological samples, cells, or exosomes can be biomarkers as well, such as lipids, carbohydrates, or poly (ADP) ribose (PAR) polymer.

Expression levels of biomarkers can be determined by any method known in the art. For example, expression levels of nucleic acid can be determined using Northern blotting, Southern blotting, microarray analysis, PCR, RT-PCR, and qPCR. Expression levels of proteins can be determined using Western blotting, enzyme-linked immunosorbent assay (ELISA), protein immunoprecipitation, immunoelectrophoresis, microarray analysis, flow cytometry, immunostaining, immunocytochemistry (ICC), immunohistochemistry (IHC), high-performance liquid chromatography (HPLC), and liquid chromatography-mass spectrometry (LC/MS), for example. Levels of protein phosphorylation can be determined using phospho-specific antibodies, kinase assays, labeling of whole cells with ³²P-orthophosphate and extract preparation, Western blotting, enzyme-linked immunosorbent assay (ELISA), cell-based ELISA, flow cytometry, ICC, IHC, mass spectrometry, and phospho-protein multiplex assays, for example. A person of skill in the art will appreciate that any other suitable method for determining expression levels and phosphorylation levels of biomarkers can be used.

Activation levels of a protein or enzyme biomarker can correlate with the level of phosphorylation. In some embodiments, activation levels of a protein or enzyme biomarker are determined by measuring phosphosylation levels using any of the methods described above. Depending on the protein or enzyme, increased phosphorylation can result in activation or inactivation of the protein or enzyme. An example of phosphorylation resulting in activation of the protein is phosphorylation of c-Abl, as described further below. An example of phosphorylation leading to inactivation is phosphorylation of parkin, as described further below.

Activation levels of a protein or enzyme biomarler can be determined by measuring the activity of the protein or enzyme in activity assays, for example. Exemplary activity assays include kinase assays, phosphatase assays, protease and peptidase assays, lipase and phospholipase assays, and others.

Activation levels of a protein or enzyme biomarker can also be determined by measuring expression and/or phosphorylation levels of molecules or substrates downstream of the protein or enzyme. As an example, to measure activation of c-Abl, phosphorylation of parkin can be measured, as described further below.

In some embodiments, one or more biomarker used in the methods of the invention is a nucleic acid. In some embodiments, one or more biomarker used in the methods of the invention is a protein or enzyme. In some embodiments, one or more biomarker used in the methods of the invention is a lipid, a carbohydrate, or a polymer such as PAR. In some embodiments, one or more biomarker used in the methods of the invention is a combination of a protein or enzyme, a nucleic acid, a lipid, a carbohydrate, or any other polymer such as PAR, for example.

### c-Abl Pathway

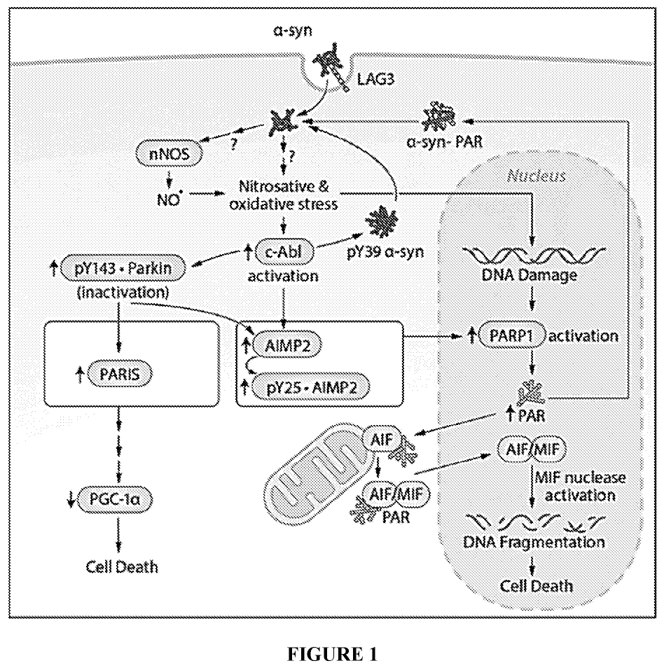

In some embodiments, one or more biomarker used in the methods of the present invention is a molecule of the c-Abl pathway (FIG. 1). Any molecule of the c-Abl pathway can be a biomarker used in the methods described herein.

In accordance with some embodiments, one or more biomarker of the c-Abl pathway used in the methods described herein is c-Abl. In some embodiments, one or more biomarker of the c-Abl pathway used in the methods described herein is α-synuclein. In some embodiments, one or more biomarker of the c-Abl pathway used in the methods described herein is parkin. In some embodiments, one or more biomarker of the c-Abl pathway used in the methods described herein is AIMP2. In some embodiments, one or more biomarker of the c-Abl pathway used in the methods described herein is PARIS (ZNF746). In some embodiments, one or more biomarker of the c-Abl pathway used in the methods described herein is PARP1. In some embodiments, one or more biomarker of the c-Abl pathway used in the methods described herein is PAR. Any combination of molecules of the c-Abl pathway can be biomarkers in the methods described herein. For example, any combination of c-Abl, α-synuclein, parkin, AIMP2, PARIS (ZNF746), PARP1, and PAR can be used as biomarkers. Additional biomarkers can include LAG3, nNOS, PGC1α, AIF, MIF (FIG. 1), either alone or in combination, or in combination with c-Abl, α-synuclein, parkin, AIMP2, PARIS (ZNF746), PARP1, and PAR, for example.

The Poly (ADP-ribose) polymerase (PARP) family comprises at least 17 family members. PARP family members can have confirmed or putative PARP activity. PARP family members can function in DNA repair, apoptosis, necrosis, transcriptional regulation, inflammation, and chromatin modification, for example. PARP family members include PARP1, PARP2, PARP3, VPARP (PARP4), Tankyrase-1 and -2 (PARP-5a or TNKS, and PARP-5b or TNKS2), PARP6, TIPARP (PARP7), PARP8, PARP9, PARP10, PARP11, PARP12, PARP13, PARP14, PARP15, and PARP16. Any PARP family member can be a biomarker in the methods described herein. PARP family members can be biomarkers either alone or in combination, or in combination with any other biomarker described herein. In some embodiments, a PARP family member is a biomarker of the c-Abl pathway. In some embodiments, a PARP family member is a biomarker of a DNA repair, apoptosis, necrosis, transcriptional regulation, inflammation, or chromatin modification pathway, or any combination thereof. In some embodiments, a PARP family member is a biomarker of the c-Abl pathway and a biomarker of one or more of a DNA repair, apoptosis, necrosis, transcriptional regulation, inflammation, or chromatin modification pathway.

As described above, the expression level, the phoshphorylation level, and/or the activation level of biomarkers used in the methods described herein can be altered in a neurodegenerative disease relative to the the expression level, the phosphorylation level, and/or the activation level of biomarkers in the absence of the neurodegenerative disease. The expression level, the phoshphorylation level, and/or the activation level of biomarkers used in the methods described herein can be also altered upon progression of the neurodegenerative disease and when sampled at a later time point relative to an earlier time point of the neurodegenerative disease. For example, the level of c-Abl phosphorylation can be increased, the level of α-synuclein phosphorylation can increased, the level of parkin phosphorylation can be increased, the expression level of AIMP2 can be increased, the level of AIMP2 phosphorylation can be increased, the expression level of PARIS (ZNF746) can be increased, the level of PARP1 activation can be increased, and the level or PAR can be increased. In some embodiments, the altered expression level, the altered phosphorylation level, and/or the altered activation level of the biomarker of the c-Abl pathway can be any combination of increased c-Abl phosphorylation level, increased α-synuclein phosphorylation level, increased parkin phosphorylation level, increased AIMP2 expression level, increased AIMP2 phosphorylation level, increased PARIS (ZNF746) expression level, increased PARP1 activation level, and increased level of PAR.

Phosphorylation of proteins typically occurs on tyrosine, serine, and threonine residues. Other residues that can be phosphorylated include histidine, arginine, lysine, aspartic acid, glutamic acid, and cysteine, for example. Any residue of the biomarkers of the c-Abl pathway that can be phosphorylated can have increased or decreased phosphorylation in the methods described herein. In some embodiments, phosphorylation of c-Abl is selected from tyrosine 245 (Y245); tyrosine 412 (Y412); and tyrosine 245 (Y245) and tyrosine 412 (Y412). In some embodiments, phosphorylation of α-synuclein is selected from tyrosine 39 (Y39); serine 129 (S129); and tyrosine 39 (Y39) and serine 129 (S129). In some embodiments, phosphorylation of parkin is on tyrosine 143 (Y143). In some embodiments, phosphorylation of AIMP2 is on tyrosine 25 (Y25). In some embodiments, phosphorylation comprises any combination of: (a) phosphorylation of c-Abl is selected from tyrosine 245 (Y245); tyrosine 412 (Y412); and tyrosine 245 (Y245) and tyrosine 412 (Y412); (b) phosphorylation of α-synuclein is selected from tyrosine 39 (Y39); serine 129 (S129); and tyrosine 39 (Y39) and serine 129 (S129); (c) phosphorylation of parkin is on tyrosine 143 (Y143); and (d) phosphorylation of AIMP2 is on tyrosine 25 (Y25).

### Neurodegenerative Diseases

The methods described herein can be used to diagnose neurodegenerative diseases, monitor progression of neurodegenerative diseases, and treat neurodegenerative diseases. Neurodegenerative diseases are disorders that destroy motor neurons or their function, for example. The methods described herein can be applied to any neurodegenerative disease. Exemplary neurodegenerative diseases include synucleinopathies, tauopathies, prion diseases, motor neuron diseases, dementia, transmissible spongiform encephalopathies, systemic atrophies primarily affecting the central nervous system, trinucleotide repeat disorders, proteopathies, amyloidosis, neuronal ceroid lipofuscinoses, and others.

In some embodiments, the methods described herein are used to diagnose a synucleinopathy, monitor progression of a synucleinopathy, or treat a synucleinopathy. Synucleinopathies are characterized by the abnormal accumulation of aggregates of α-synuclein in neurons, nerve fibers, or glial cells. Exemplary synucleinopaties include Parkinson's disease, dementia with Lewy bodies, multiple system atrophy (MSA), and certain neuraxonal dystrophies. In some embodiments, the synucleinopathy is Parkinson's disease.

The methods described herein include determining expression levels, phosphorylation levels, and/or activation levels of biomarkers in a biological sample obtained from a subject having a neurodegenerative disease or suspected of having a neurodegenerative disease and in biological samples obtained from control subjects. In some embodiments, the control subject is healthy. In some embodiments, the control subject does not suffer from a neurodegenerative disease. In some embodiments, the control subject suffers from forms of Parkinsonism other than Parkinson's disease. In some embodiments, the control subject suffers from Parkinsonism. In some embodiments, the control subject suffers from Parkinson's-like disease. In some embodiments, the control subject suffers from a tauopathy.

The methods described herein to diagnose neurodegenerative diseases, monitor progression of neurodegenerative diseases, and treat neurodegenerative diseases can be used to determine that the neurodegenerative disease is a synucleinopathy as distinghushed from a tauopathy. Tauopathies are neurodegenerative diseases associated with the pathological aggregation of tau protein in neurofibrillary or gliofibrillary tangles in the human brain. Exemplary tauopathies include, but are not limited to, Alzheimer's disease, primary age-related tauopathy (PART), chronic traumatic encephalopathy (CTE), progressive supranuclear palsy (PSP), corticobasal degeneration (CBD), frontotemporal dementia and parkinsonism linked to chromosome 17 (FTDP-17), amyotrophic lateral sclerosis-parkinsonism-dementia (ALS-PDC, Lytico-bodig disease), ganglioglioma, gangliocytoma, meningioangiomatosis, postencephalitic parkinsonism, subacute sclerosing panencephalitis (SSPE), lead encephalopathy, tuberous sclerosis, pantothenate kinase-associated neurodegeneration, and lipofuscinosis.

### Treatment of Neurodegenerative Disorders

In some embodiments, the methods described herein comprise providing a report of an altered expression level, an altered phosphorylation level, and/or an altered activation level of one or more biomarkers for selection of a treatment of a subject suffering from a neurodegenerative disease or suspected of suffering from a neurodegenerative disease. Selection of a treatment can include admininstering a therapeutic agent to the subject. The therapeutic agent can be a small molecule, a drug, an antibody, a hybrid antibody, an antibody fragment, an siRNA, an antisense RNA, an aptamer, a protein, or a peptide. Selection of a treatment can also include not treating the subject, making changes to a treatment the subject is already receiving, and continuing the same treatment, i.e., not making changes to a treatment the subject is already receiving.

In some embodiments, the methods described herein comprise treating a neurodegenerative disorder in a subject. Treating a neurodegenerative disorder can comprise administration of a therapeutic agent to a subject. The therapeutic agent can be a small molecule, a drug, an antibody, a hybrid antibody, an antibody fragment, an siRNA, an antisense RNA, an aptamer, a protein, or a peptide. In some embodiments, the therapeutic agent is an inhibitor of the c-Abl pathway. In some embodiments, the inhibitor of the c-Abl pathway is a kinase inhibitor. Kinase inhibitors include tyrosine kinase inhibitors and serine/threonine kinase inhibitors, for example. In some embodiments, the kinase inhibitor is a c-Abl inhibitor. Exemplary c-Abl inhibitors include nilotinib, K0706, Imatinib (Gleevec®), PD180970, PD166325, PD173955, Radotinib HCl, IkT-001Pro, IkT-148x, IkT-1427, and IkT-148009. In some embodiments, the inhibitor of the c-Abl pathway is a PARP inhibitor. In some embodiments, the PARP inhibitor is a PARP1 inhibitor. Inhibitors of any PARP family member can be used in the methods described herein, including inhibitors of PARP1, PARP2, PARP3, VPARP (PARP4), Tankyrase-1 and -2 (PARP-5a or TNKS, and PARP-5b or TNKS2), PARP6, TIPARP (PARP7), PARP8, PARP9, PARP10, PARP11, PARP12, PARP13, PARP14, PARP15, and PARP16, for example. Exemplary PARP inhibitors include Olaparib, Rucaparib, Niraparib, Talazoparib, Veliparib (ABT-888), BGB-290 (Pamiparib), CEP 9722, E7016, Iniparib (BSI 201), 3-Aminobenzamide, AG-14361, A-966492, PJ34 HCl, UPF 1069, ME0328, NMS-P118, E7449, Picolinamide, Benzamide, NU1025, AZD2461, BGP-15 2HCl.

### EXAMPLES

### Example 1

This example illustrates that L1CAM+ exosomes contain neuronal molecules or markers.

Exosomes are extracellular vesicles that contain within their lipid bilayer membrane specific molecules, allowing for identification of their cellular origins. Neuronal cells secrete L1CAM+ exosomes, which can be isolated from the serum. Purification of the L1CAM+ exosome from the serum therefore allows for identification of neuronal cellular content coupled with all of the benefits of the exosome being blood-based. Excitingly, as described below, it has been possible to (a) detect each of the c-Abl pathway proteins (FIG. 1) discussed below in the L1CAM+ exosome and (b) find differences in relative concentrations of these molecules between Parkinson's disease (PD) patients and age-matched controls, and (c) find differences in relative concentrations of these molecules between PD patients and individuals with other forms of parkinsonism. These molecules are highly predictive, selective, and specific biomarkers for PD that can used for the clinical diagnosis, monitoring progression of the disorder or as theranostic markers.

### Example 2

This example illustrates levels of c-Abl phosphorylation at Y245 in neuronal exosomes in Parkinson's disease and other synucleinopathies.

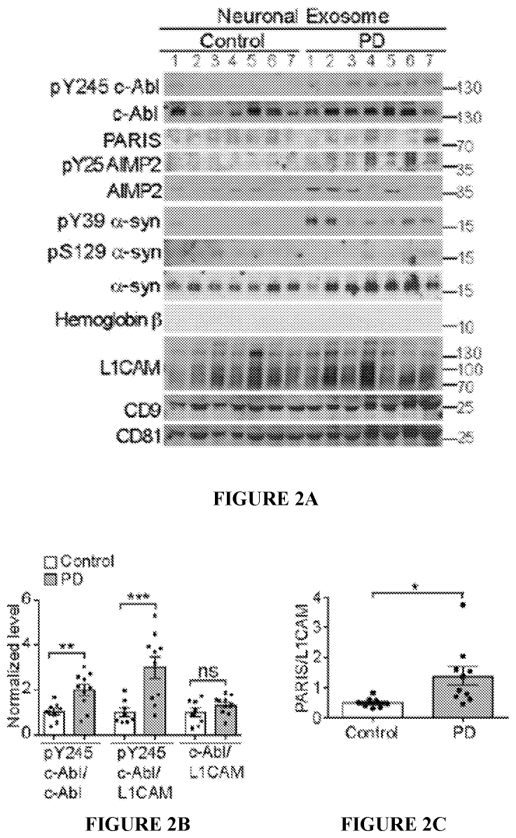

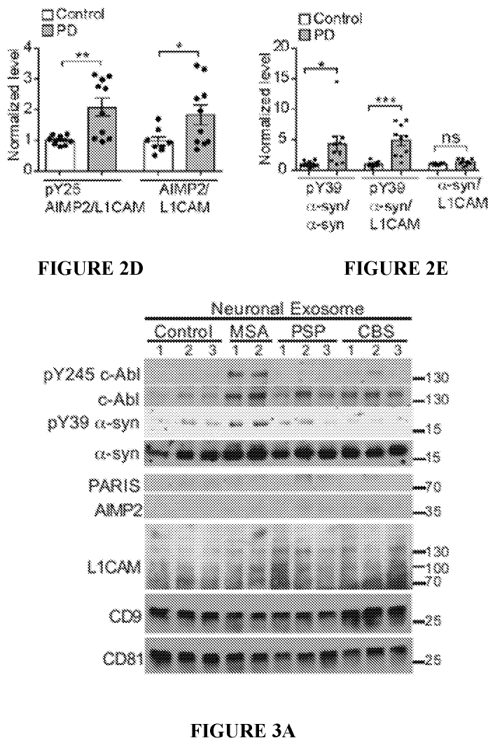

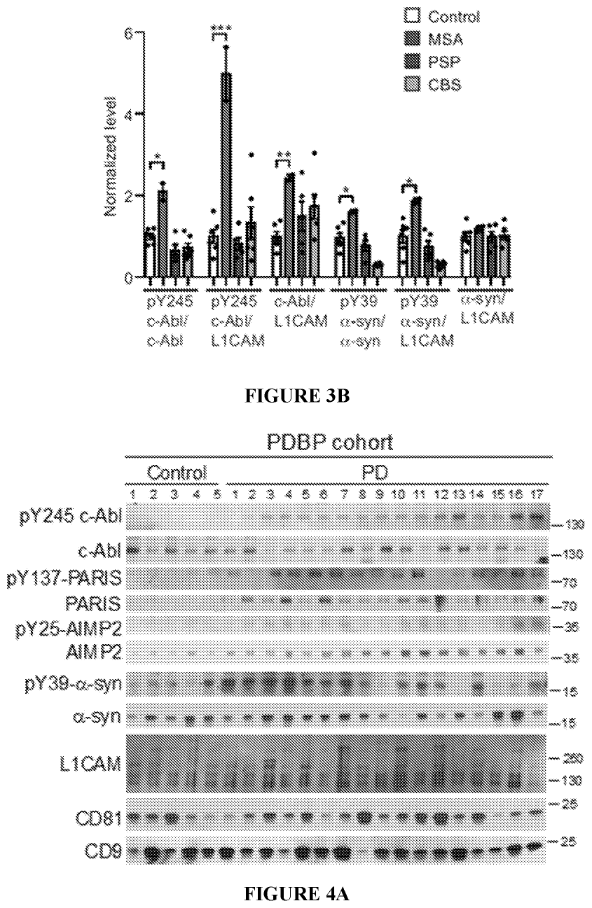

The activation of c-Abl is a first step of the c-Abl pathway (FIG. 1). c-Abl undergoes both autophosphorylation leading to its activation and enhancement of the activated levels in situations of oxidative stress. When c-Abl is activated it is phosphorylated at two tyrosine sites, Y412 and Y245 (FIG. 2). Without being limited by theory, phosphorylation at both sites may be necessary for complete activation of c-Abl, with Y412 phosphorylation occurring first in the cascade followed by Y245 phosphorylation. Identification and levels of the Y245 c-Abl within the L1CAM+ exosome was focused on because phosphorylation at that site indicates complete activation of the kinase. Without being limited by theory, pY412 should also serve a similar role as a biomarker of the c-Abl pathway. As discussed further below, data shown in FIG. 2 indicates greater c-Abl phosphorylation at Y245 among individuals with Parkinson's disease (PD) and other synucleinopathies than among controls and the tauopathies. The c-Abl activation led to inactivation of parkin, as shown by accumulation of PARIS and AIMP2 (FIGS. 2 and 3; Example 3, below) and phosphorylation of α-syn at Y39 (FIG. 3; Example 4, below).

These data show that neuronal exosomes contain c-Abl, with greater phosphorylation of c-Abl at Y245 in Parkinson's disease (PD) and other synucleinopathies as compared to control and tauopathies. These results establish phosphorylation of c-Abl at Y245 as a biomarker for PD.

### Example 3

This example illustrates PARIS and AIMP2 levels in L1CAM+ exosomes.

Without being limited by theory, loss of parkin function leads to accumulation of parkin interacting substrate (PARIS) and aminoacyl-tRNA synthetase-interacting multifunctional protein type 2 (AIMP2). PARIS contains a Kruppel-Associated Box (KRAB), a zinc-finger at its C-terminus and is highly preserved across species. Parkin regulates PARIS through ubiquitination, thus marking PARIS for subsequent clearance. Without being limited by theory, when PARIS is not ubiquitinated by parkin, PARIS accumulates and ultimately leads to neuronal cell death. The ubiquitination of PARIS is dependent on phosphorylation at two specific phosphorylation sites.

PARIS was detected in L1CAM+ positive serum exosomes and its level was elevated in PD, but not in tauopathies, which is indicative of its potential as a biomarker candidate (FIG. 2 and FIG. 3). Similar evidence supports the role of AIMP2 increase in PD pathophysiology (FIG. 2). Without being limited by theory, AIMP2 overexpression results in an age-dependent degeneration of dopaminergic (DA) neurons and AIMP2 transgenic mice exhibit neuronal degeneration solely in DA neurons in the substantia nigra. Furthermore, a novel tyrosine phosphorylation site of AIMP2 (Y25) was identified, with phosphorylation mediated by c-Abl (FIG. 2). Both phosphorylated Y25 AIMP2 and unphosphorylated AIMP2 were detected in L1CAM+ exosomes, with higher levels of phosphorylated AIMP2 in PD than controls and tauopathies (FIG. 2 and FIG. 3).

These data show that PARIS and AIMP2 accumulate in neuronal exosomes in PD, but not in tauopathies. Thus, the data support a role of PARIS and AIMP2 as biomarkers for PD and related synucleinopathies.

### Example 4

This example describes c-Abl phosphoryolation of α-syn at Y39.

Without being limited by theory, α-syn is thought to play an important role in PD pathogenesis. The primary component of Lewy Bodies (LBs), the pathological hallmark of PD (in both idiopathic and familial forms), has been identified as aggregated α-syn. Without being limited by theory, α-syn alterations and post-translational modifications (PTMs) have been proposed as early steps of Lewy body formation. A unique subset of α-syn phosphorylation is associated with Lewy bodies, including phosphorylation at Y39 and S129, indicating that specific α-syn posttranslational modifications may participate in the pathogenesis of PD. Data in FIG. 2 and FIG. 3 shows that phosphorylation of Y39 is elevated in PD, MSA, but not in control or tauopathy subjects.

These data support phosphorylation of Y39 of α-syn, a component of the c-Abl pathway, as a biomarker for PD and related synucleinopathies.

### Discussion of Examples 1-4

### c-Abl Pathway Molecules are Suitable Biomarkers

Without being limited by theory, activation of the non-receptor stress activated tyrosine kinase c-Abl in PD leads to a downstream cascade of changing cellular and molecular activity and ultimately cell death of dopaminergic (DA) neurons. Evidence suggests that aberrant activation of c-Abl may play a role in the pathogenesis of PD. c-Abl pathway molecules can serve as biomarkers (FIG. 1). Current evidence indicates that tyrosine phosphorylated c-Abl is elevated in the substantia nigra and striatum in the brains of PD patients and suggests that c-Abl is activated as part of PD pathogenesis, while it is not elevated in other brain regions that do not exhibit substantial pathology. Moreover c-Abl knockout (KO) or c-Abl inhibitors protect against the loss of DA neurons in the substantia nigra of MPTP-intoxicated mice. c-Abl KO slows the progression of human A53T α-syn transgenic mice and reduces the neuropathology and behavioral deficits. In addition, c-Abl KO or c-Abl inhibitors prevent the loss of DA neurons in the α-syn preformed fibril (PFF) model of PD.

Data shown in Examples 1-4 coupled with other studies supporting the role of c-Abl activation in PD has prompted large, phase II randomized control trials testing the safety of the c-Abl inhibitors, nilotinib and K0706, in a PD population. Without being limited by theory, a hypothesis is that pathologic α-syn activates c-Abl through as yet uncharacterized mechanisms. Activated (phosphorylated) c-Abl, as measured via the levels of pY245 c-Abl, then phosphorylates α-syn at Y39 and parkin at Y143. Without being limited by theory, it is possible that phosphorylation of α-syn at Y39 leads to a more toxic form of α-syn. Phosphorylation of parkin at Y143 leads to its inactivation and accumulation of AIMP2 and PARIS (ZNF746), which contribute to the demise of DA neurons. Ultimately, elevation of AIMP2 and its phosphorylation of Y25 contribute to activation of PARP1, leading to an increase in the PAR polymer. Thus, c-Abl, α-syn, PARIS and AIMP2 and tyrosine phosphorylated α-syn and AIMP2 can serve as markers of c-Abl activation. These molecules from the c-Abl pathway and other yet to be identified c-Abl substrates can serve as specific and selective markers of PD. By examining the multiple components of the c-Abl pathway in the L1CAM+ neuronal exosomes, it has been determined that these molecules are diagnostic for PD and may also be progression markers, as shown by data in Examples 1-4.

### Example 5

### Evaluation of c-Abl Pathway Molecules in Serum-Derived L1cam+ Exosomes

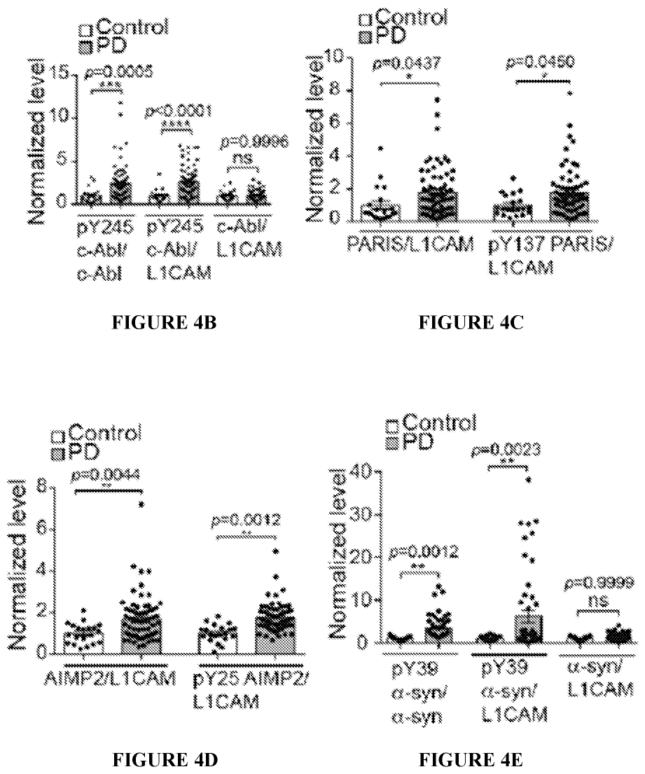

Serum-derived L1CAM+ exosomes were isolated from a PD patient and analyzed for their expression of pY245 c-Abl, c-Abl, pY137 PARIS, PARIS, pY25 AIMP2, AIMP2, pY39 α-syn, α-syn, L1CAM, CD81, and CD9. As illustrated in FIG. 4A, immunoblots of pY245 c-Abl, c-Abl, pY137 PARIS, PARIS, pY25 AIMP2, AIMP2, pY39 α-syn, α-syn, L1CAM, CD81, and CD9 show that molecules of c-Abl pathway were elevated in serum-derived L1CAM+ exosomes from PD patients as compared to control subjects.

As detailed in FIGS. 4B, 4C, 4D and 4E, the results were quantified and normalized. pY245 c-Abl levels were normalized to c-Abl and to L1CAM (FIG. 4B); PARIS and pY137 levels were normalized to L1CAM (FIG. 4C); AIMP2 and pY25 AIMP2 levels were normalized to L1CAM (FIG. 4D); and pY39 α-syn levels were normalized to α-syn and to L1CAM (FIG. 4E); confirming the upregulation of those markers in PD patients as compared to controls. Normalization was performed with n=30 controls, and n=77 PD. Statistical significance was determined by one-way ANOVA with Sidak's post-test of multiple comparison. Quantified data were expressed as mean±s.e.m.

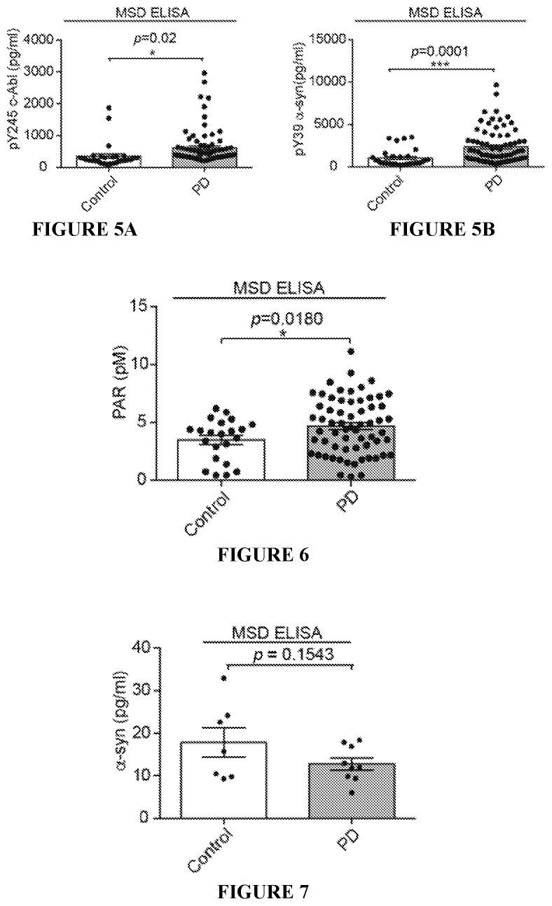

Electrochemiluminescence (ECL)-based quantitative immunoassays were then performed to quantify pY245 c-Abl, pY39 α-synuclein, PAR, and α-synuclein in serum-derived L1CAM+ exosomes of PD patients. As illustrated in FIGS. 5A and 5B, MSD-based quantitative immunoassay shown elevation of pY245 c-Abl and pY39 α-synuclein in serum-derived L1CAM+ exosomes of PD patients as compared to controls. Electrochemiluminescence (ECL)-based quantitative immunoassays for pY245 c-Abl and pY39 α-syn were performed on MSD (Meso-scale Discovery) Quickplex SQ120 platform using L1CAM+ exosome lysates from control and PD patients (Control, n=30; PD, n=77). The bar diagrams represent concentrations of pY245 c-Abl and pY39 α-syn. Statistical significance was determined by unpaired two-tailed t-test. Quantified data are expressed as mean±s.e.m.

As illustrated in FIG. 6, MSD-based quantitative immunoassay revealed increased PAR levels in serum-derived L1CAM+ exosomes of PD patients as compared to controls. ECL immunoassay for PAR was performed on MSD Quickplex SQ120 electrochemiluminescence platform using L1CAM+ exosome lysates from control and PD patients (Control, n=22; PD, n=63). The bar diagram represents concentrations of PAR. Statistical significance was determined by unpaired two-tailed t-test. Quantified data are expressed as mean±s.e.m. Further, as shown in FIG. 7, MSD-based quantitative immunoassay for α-synuclein in serum-derived L1CAM+ exosomes of PD cases and age-matched healthy controls. ECL immunoassay for α-synuclein was performed on MSD Quickplex SQ120 electrochemiluminescence platform using L1CAM+ exosome lysates from control and PD patients (Control, n=7; PD, n=9). The bar diagram represents concentrations of α-synuclein. Statistical significance was determined by unpaired two-tailed t-test. Quantified data are expressed as mean±s.e.m.

### Example 6

### Characterization of Serum-Derived Exosomes

Serum-derived neuronally enriched exosomes were isolated by polymer-based nanoparticle precipitation followed by an immunoprecipitation method, and were characterized.

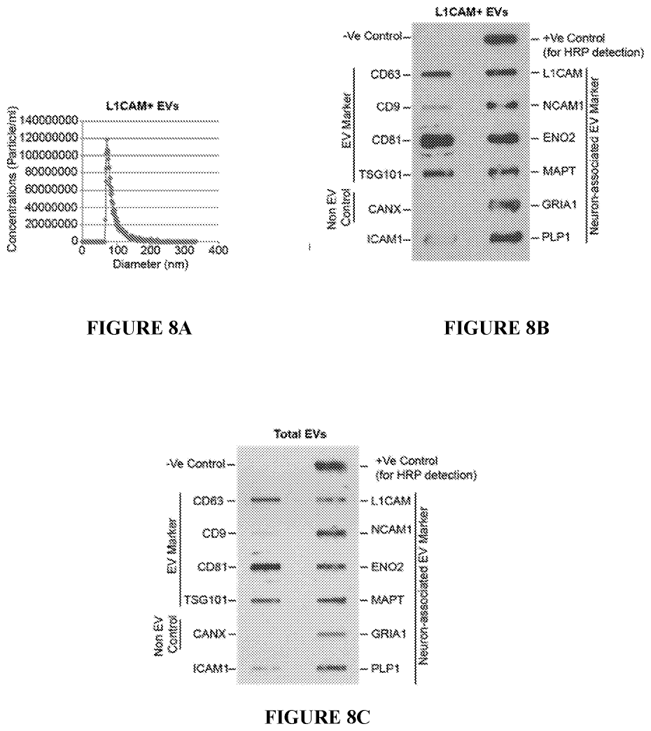

The concentrations and sizes of serum-derived L1CAM-enriched EVs was analyzed by Spectradyne's nCS1, using a combination of microfluidic and nanotechnology to detect and measure every particle in the formulation. As illustrated in FIG. 8A, the particle size of the extracellular vesicles under the peak ranged from 66 to 130 nm, which is consistent with the sizes of exosomes. Exo-Check Exosome Antibody Array (Neuro) standard kit from SBI was used to evaluate the canonical exosomal markers and the neuronal markers in the L1CAM-enriched neuronal EVs (FIG. 8B) and CD81+ total EVs (FIG. 8C).

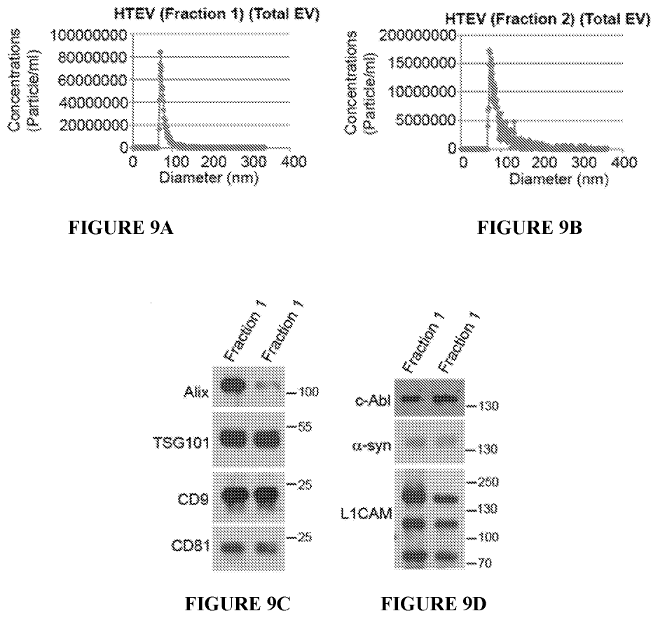

Serum derived total exosomes were further isolated by a high throughput size-exclusion-based EV isolation method and characterized. As illustrated in FIGS. 9A and 9B, the concentrations and sizes of serum-derived EV fraction 1 (FIG. 9A) and fraction 2 (FIG. 9B) were analyzed by Spectradyne's nCS1. The particle sizes under the peak were consistent with the dimensions of EVs. As shown in the immunoblots illustrated in FIG. 9C, the canonical exosomal markers Alix, TSG101, CD9, and CD81 were analyzed, and found expressed in fractions 1 and 2 of EV lysates. As shown in the immunoblots illustrated in FIG. 9D c-Abl, α-synuclein, and the neuronal marker L1CAM were found expressed in fraction 1 and 2 of EV lysates.

Any and all references and citations to other documents, such as patents, patent applications, patent publications, journals, books, papers, web contents, that have been made throughout this disclosure are hereby incorporated herein by reference in their entirety for all purposes.

Although the present invention has been described with reference to specific details of certain embodiments thereof in the above examples, it will be understood that modifications and variations are encompassed within the spirit and scope of the invention. Accordingly, the invention is limited only by the following claims.

1. A method of diagnosing a neurodegenerative disease in a subject comprising:
(a) determining an expression level, a phosphorylation level, and/or an activation level of one or more biomarkers in a biological sample obtained from a subject suspected of having a neurodegenerative disease;
(b) determining that the subject suffers from a neurodegenerative disease when the expression level, the phosphorylation level, and/or the activation level of the one or more biomarkers in the biological sample is altered relative to the expression level, the phosphorylation level, and/or the activation level of the one or more biomarkers in a biological sample of a control subject; and
(c) providing a report of the determination that the subject suffers from a neurodegenerative disease for selection of a treatment of the subject,
thereby diagnosing a neurodegenerative disease.

2. (canceled)

3. The method of claim 1, wherein the one or more biomarkers is a molecule of the c-Abl pathway.

4. (canceled)

5. The method of claim 3, wherein the molecule is c-Abl, α-synuclein, parkin, AIMP2, PARIS (ZNF746), PARP1, PAR, or a combination thereof.

6. The method of claim 1, wherein the altered expression level, the altered phosphorylation level, and/or the altered activation level of the one or more biomarkers is selected from the group consisting of:
(a) the level of c-Abl phosphorylation is increased;
(b) the level of c-Abl activation is increased;
(c) the level of α-synuclein phosphorylation is increased;
(d) the level of parkin phosphorylation is increased;
(e) the level of parkin inactivation is increased;
(f) the expression level of AIMP2 is increased;
(g) the level of AIMP2 phosphorylation is increased;
(h) the expression level of PARIS (ZNF746) is increased;
(i) the level of PARP1 activation is increased; and
(j) the level of PAR is increased;
or a combination thereof.

7-10. (canceled)

11. The method of claim 6, wherein phosphorylation comprises:
(a) phosphorylation of c-Abl selected from the group consisting of tyrosine 245 (Y245); tyrosine 412 (Y412); and tyrosine 245 (Y245) and tyrosine 412 (Y412);
(b) phosphorylation of α-synuclein selected from the group consisting of tyrosine 39 (Y39); serine 129 (S129); and tyrosine 39 (Y39) and serine 129 (S129);
(c) phosphorylation of parkin on tyrosine 143 (Y143);
(d) phosphorylation of AIMP2 on tyrosine 25 (Y25);
or any combination thereof.

12. The method of claim 1, wherein the biological sample is a blood sample, a plasma sample, or a serum sample.

13. The method of claim 12, wherein the biological sample comprises exosomes.

14. (canceled)

15. The method of claim 13, wherein the neuronal marker is L1CAM.

16. The method of claim 1, wherein the neurodegenerative disease is a synucleinopathy.

17. The method of claim 16, wherein the synucleinopathy is Parkinson's disease, dementia with Lewy bodies, multiple system atrophy (MSA), or a neuraxonal dystrophy.

18-19. (canceled)

20. A method of monitoring progression of a neurodegenerative disease in a subject comprising:
(a) determining an expression level, a phosphorylation level, and/or an activation level of one or more biomarkers in a biological sample obtained from a subject suspected of having a neurodegenerative disease;
wherein the expression level, the phosphorylation level, and/or the activation level of the one or more biomarkers is altered upon progression of the neurodegenerative disease and when sampled at a later time point relative to an earlier time point of the neurodegenerative disease; and
(b) providing a report of the altered expression level, the altered phosphorylation level, and/or the altered activation level of the one or more biomarkers for selection of a treatment of the subject,
thereby monitoring progression of the neurodegenerative disease.

21. (canceled)

22. The method of claim 20, wherein the one or more biomarkers is a molecule of the c-Abl pathway.

23. (canceled)

24. The method of claim 22, wherein the molecule is c-Abl, α-synuclein, parkin, AIMP2, PARIS (ZNF746), PARP1, PAR, or a combination thereof.

25. The method of claim 20, wherein the altered expression level, the altered phosphorylation level, and/or the altered activation level of the one or more biomarkers is selected from the group consisting of:
(a) the level of c-Abl phosphorylation is increased;
(b) the level of c-Abl activation is increased;
(c) the level of α-synuclein phosphorylation is increased;
(d) the level of parkin phosphorylation is increased;
(e) the level of parkin inactivation is increased;
(f) the expression level of AIMP2 is increased;
(g) the level of AIMP2 phosphorylation is increased;
(h) the expression level of PARIS (ZNF746) is increased;
(i) the level of PARP1 activation is increased; and
(j) the level of PAR is increased;
or a combination thereof.

26-29. (canceled)

30. The method of claim 25, wherein phosphorylation wherein phosphorylation comprises:
(a) phosphorylation of c-Abl selected from the group consisting of tyrosine 245 (Y245); tyrosine 412 (Y412); and tyrosine 245 (Y245) and tyrosine 412 (Y412);
(b) phosphorylation of α-synuclein selected from the group consisting of tyrosine 39 (Y39); serine 129 (S129); and tyrosine 39 (Y39) and serine 129 (S129);
(c) phosphorylation of parkin on tyrosine 143 (Y143);
(d) phosphorylation of AIMP2 on tyrosine 25 (Y25);
or any combination thereof.

31. The method of claim 20, wherein the biological sample is a blood sample, a plasma sample, or a serum sample.

32. The method of claim 31, wherein biological sample comprises exosomes.

33-34. (canceled)

35. The method of claim 20, wherein the neurodegenerative disease is a synucleinopathy selected from the group consisting of Parkinson's disease, dementia with Lewy bodies, multiple system atrophy (MSA), and a neuraxonal dystrophy.

36. (canceled)

37. The method of claim 20, wherein the expression level, the phosphorylation level, and/or the activation level of the one or more biomarkers is determined at two or more time points.

38. A method of treating a neurodegenerative disease in a subject comprising administering an inhibitor of the c-Abl pathway to the subject when the expression level, the phosphorylation level, and/or the activation level of one or more biomarkers of the c-Abl pathway in a biological sample obtained from the subject is altered relative to the expression level, the phosphorylation level, and/or the activation level of the one or more biomarkers in a biological sample of a control subject, thereby treating the neurodegenerative disease.

39-58. (canceled)
